(19) United States
(12) Patent Application Publication
(10) Pub. No.: US 2022/0284403 A1
(43) Pub. Date: Sep. 8, 2022

(54) System and Method for Providing Cryotherapy Services

(71) Applicant: Gregory Papageorge, Old Westbury, NY (US)

(72) Inventor: Gregory Papageorge, Old Westbury, NY (US)

(21) Appl. No.: 17/509,377

(22) Filed: Oct. 25, 2021

Related U.S. Application Data
(60) Provisional application No. 63/155,519, filed on Mar. 2, 2021.

(51) Int. Cl. G06Q 10/10; G16H 10/60; G16H 20/30

(52) U.S. Cl. CPC G06Q 10/1095; G16H 10/60; G16H 20/30

(57) ABSTRACT

The present invention relates to a system and method for purchase or booking sessions of cryotherapy as per the preferences of a user using a website and software application hosted on a server. A cryotherapy session request from a patient is received at the software application. The request is transmitted to the server via a network. The server allocates a cryotherapy machine or device and an operator for performing a cryotherapy session at a scheduled place (i.e. remote location) and time. The system allows a patient to select a treatment type, a location, and a time for the cryotherapy session.

### CROSS-REFERENCE TO RELATED APPLICATION

The present application claims priority to, and the benefit of, U.S. Provisional Application No. 63/155,519, which was filed on Mar. 2, 2021 and is incorporated herein by reference in its entirety.

### FIELD OF THE INVENTION

The present invention relates generally to the field of systems and methods for booking appointments. More specifically, the present invention relates to a system and method for booking cryotherapy/cold therapy unit or services, that provides access to portable cryotherapy machine/session at a desired time slot. The cryotherapy machines soothe muscle aches, reduces inflammation, relieves chronic pain, boosts metabolism and promotes healing and weight loss. Individuals can easily use the software application or can alternatively connect to the cryotherapy session booking system using a website, for booking an appointment or machine for a specific issue. Accordingly, the present disclosure makes specific reference thereto. Nonetheless, it is to be appreciated that aspects of the present invention are also equally applicable to other like applications, devices, and methods of manufacture.

### BACKGROUND OF THE INVENTION

By way of background, individuals suffering from muscle aches and pains, inflammation, chronic pain, etc. may be looking for different ways to obtain comfort and relief. Cryotherapy is a low-temperature treatment used to repair muscles, promote healthy fiber restoration, and minimize post-workout fatigue. In cryotherapy, also referred as “cold therapy”, extreme cold is used to freeze and remove abnormal tissue. Doctors also use this therapy for treating many skin conditions (such as warts and skin tags) and some cancers, including prostate, cervical and liver cancer. Machines or devices such as cryotherapy saunas, cryotherapy chambers, localized cryotherapy machines and more are used to deliver very cold carbon dioxide to the affected area in order to soothe muscle aches, reduce inflammation, relieve chronic pain and more.

These cryotherapy-providing machines are generally available at rehabilitation centers, physiotherapy centers, healthcare centers or the like. Individuals cannot easily access these machines unless an appointment for a cryotherapy session is booked at the healthcare centers or the like, and individuals then are required to visit the healthcare center. Individuals suffering from muscle aches, post-workout fatigue and other similar issues may not be comfortable in visiting healthcare centers in order to get cryotherapy medical treatments. Individuals may be reluctant in getting therapies to treat their muscle pains and other similar issues, due to the inconvenience of travelling to healthcare centers, waiting for their appointment and more. In case the healthcare center is distant from the home of the user, it may take a lot of travelling time to travel to a healthcare center and get back home, which may waste time of the users. Additionally, these cryotherapy-providing devices are quite expensive and individuals may lack the means to easily access cryotherapy services.

Therefore, there exists a long felt need in the art for a system providing access to cryotherapy sessions while being at home. There is also a long felt need in the art for a method for providing easy access to cryotherapy machines without visiting any healthcare centers, rehabilitation centers, physiotherapy centers or the like. Additionally, there is a long felt need in the art for a solution for allowing the users to easily access cryotherapy sessions without travelling to healthcare centers and prevents them from wasting their time in travelling as well. Moreover, there is a long felt need in the art for a cryotherapy access method that eliminates the need to buy expensive cryotherapy machines to obtain access to the cryotherapy sessions at home. Further, there is a long felt need in the art for a solution that ensures individuals can conveniently access cryotherapy sessions to soothe muscle aches, reduce inflammation, relieve chronic pain and more. Finally, there is a long felt need in the art for a solution that offers a way to easily obtain multiple cryotherapy sessions without traveling considerable distances.

The subject matter disclosed and claimed herein, in one embodiment thereof, comprises a computer-implemented method for scheduling a cryotherapy session at a scheduled place (i.e. remote) and time. The method includes initially receiving a cryotherapy service session request by a server via a network wherein the request is received from a computer-implemented application installed in an electronic device or through a website. The received cryotherapy session service request is stored by the server, wherein the request includes patient information including name, contact details, treatment type, schedule information and more. Based on the received request for information, the server assigns an operator and a cryotherapy machine for the scheduled session and transmits the details of the operator and the machine to the electronic device of the user.

In this manner, the novel cryotherapy service-providing system of the present invention accomplishes all of the forgoing objectives and provides a relatively easy and convenient solution to enable the users to easily book cryotherapy sessions/machines and can easily access them while being at home. The cryotherapy service-providing system of the present invention is also user friendly, as it ensures the user can conveniently access cryotherapy sessions for treating muscle aches, relieving chronic pain and more, without travelling to a healthcare center, rehabilitation center, physiotherapy center or the like. The cryotherapy service-providing system of the present invention eliminates the need to travel for cryotherapy sessions and therefore saves the time of the users.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a computer-implemented method for scheduling a cryotherapy session at a scheduled place and time. The method includes: receiving, by a server associated with a cryotherapy service, a session request via a network for a cryotherapy session wherein the request is received from a computer-implemented application installed in an electronic device or through a website; storing by the server, patient information including name, contact details, treatment type and schedule information received in said session request; assigning by the server an operator and a cryotherapy machine for the scheduled session; and, transmitting details of the operator and the machine to the electronic device.

In yet another embodiment, the method further includes comparing by the server expertise of the operator with treatment requirement for assigning an operator. The treatment can include one or more of a post-operative treatment, a body sculpting treatment, a pain management treatment, a dermatologic treatment and a Frotox treatment.

In yet another embodiment of the present invention, the network for transmitting the session request can be the internet or any public network and the session request can be an encrypted request containing the patient's name, contact details, treatment type and schedule information such as scheduled address and time.

In yet another embodiment of the present invention, a system for providing an on-demand cryotherapy treatment session at a patient-desired location and time is disclosed. The system includes at least one server having a processor configured to receive a cryotherapy session request from a patient electronic device via a network. The system can further store the patient name, contact details, cryotherapy treatment type, and session schedule information, assign an operator and a cryotherapy machine based on received treatment type and session schedule information, and transmit confirmation and details of the operator and machine to the patient electronic device.

In yet another embodiment of the present invention, the system includes a patient information database for storing personal and treatment information of patients and an operator database to store expertise and feedback information of operators.

In yet another embodiment of the present invention, a computer-readable storage medium comprising instructions that are executed by a processor of a patient electronic device is disclosed. The instructions cause the processor to receive cryotherapy session details from a patient, submit a cryotherapy session request to a server and receive a notification confirming successful scheduling of said session. The session details include patient name, contact details, a cryotherapy treatment type, session schedule information, payment information and billing information.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a schematic view of one potential embodiment of a cryotherapy service system of the present invention for providing a plurality of cryotherapy service to a user in accordance with the disclosed architecture;

FIG. 2 illustrates a perspective view of one potential exemplary interface for booking cryotherapy treatments using the cryotherapy service system of the present invention in accordance with the disclosed architecture;

FIG. 3 illustrates storage of patient and operator information in a cryotherapy service provider server of the cryotherapy service system of the present invention in accordance with the disclosed architecture;

FIG. 4 illustrates a flow diagram showing exemplary steps performed by a patient for requesting cryotherapy service using the cryotherapy service system of the present invention in accordance with the disclosed architecture;

FIG. 5 illustrates a flow diagram showing steps performed by the cryotherapy service provider for providing a cryotherapy service session to a user in accordance with the disclosed architecture;

FIG. 6 illustrates a block diagram showing exemplary types of cryotherapy service sessions provided by the cryotherapy service system of the present invention in accordance with the disclosed architecture; and

FIG. 7 illustrates an example electronic device upon which the computer-implemented application of the present disclosure is implemented in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

Therefore, there exists a long felt need in the art for a system providing access to cryotherapy sessions while being at home. There is also a long felt need in the art for a method for providing easy access to cryotherapy machines without visiting any healthcare centers, rehabilitation centers, physiotherapy centers or the like. Additionally, there is a long felt need in the art for a solution for allowing the users to easily access cryotherapy sessions without travelling to healthcare centers, and prevents them from wasting their time in travelling as well. Moreover, there is a long felt need in the art for a cryotherapy access method that eliminates the need to buy expensive cryotherapy machines to obtain access to the cryotherapy sessions at home. Further, there is a long felt need in the art for a solution that ensures individuals can conveniently access cryotherapy sessions to soothe muscle aches, reduce inflammation, relieve chronic pain and more. Finally, there is a long felt need in the art for a solution that offers a way to easily obtain multiple cryotherapy sessions without traveling considerable distances.

The present invention, in one exemplary embodiment, is a novel cryotherapy service system for providing on-demand cryotherapy treatment session at a patient desired location and at a scheduled time is disclosed. The cryotherapy service system includes: at least one server having a processor configured to receive a cryotherapy session request from a patient electronic device via a network; store and save patient name information, contact details, cryotherapy treatment type and session schedule information; assign an operator and a cryotherapy machine based on received treatment type and session schedule information; and, transmit confirmation and details of the operator and machine to the patient electronic device.

Embodiments of the present disclosure take the form of computer-executable instructions, including algorithms executed by a programmable computer. However, the disclosure can be practiced with other computer system configurations as well. Certain aspects of the disclosure can be embodied in a special-purpose computer or data processor that is specifically programmed, configured or constructed to perform one or more of the computer-executable algorithms described below. Accordingly, the term “computer” as generally used herein refers to any data processor and includes Internet appliances, hand-held devices (including tablets, computers, wearable computers, cellular or mobile phones, multi-processor systems, processor-based or programmable consumer electronics, network computers, minicomputers) and the like.

While the present disclosure is described with particular respect to cryotherapy services, the present disclosure is not limited for use in any particular profession or area of expertise; rather, the systems and methods provided herein may be advantageously employed for use by other professionals or persons having particular expertise in any given field.

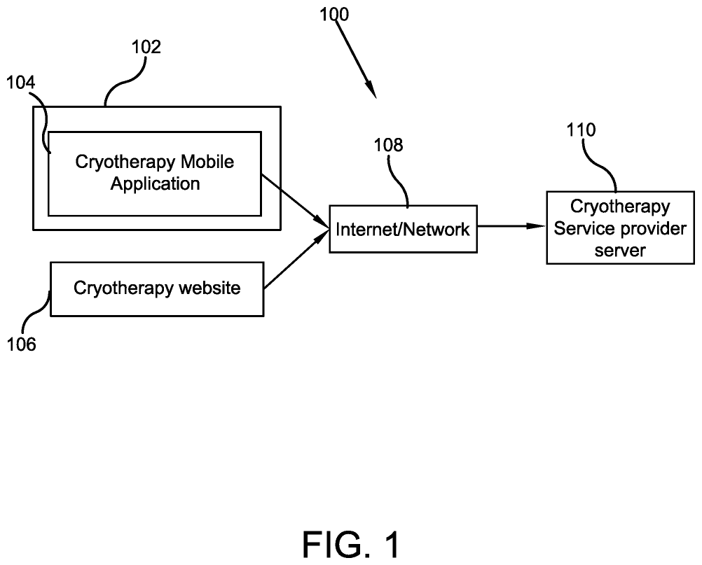

FIG. 1 illustrates a schematic view of a cryotherapy service system of the present invention for providing a plurality of cryotherapy services to a user in accordance with the disclosed architecture. The mobile cryotherapy service system 100 is designed to provide users who are suffering from muscle aches and pains, inflammation, chronic pain or any other such type of health problem with the one or more cryotherapy services sessions at a remote or home location. The cryotherapy service system 100 is designed to provide users with the portable cryotherapy machine that reduces inflammation, soothes muscle aches, relieves chronic pain, boosts metabolism and promotes healing and weight loss. The cryotherapy service system 100 is defined for providing cryotherapy services in the present invention. However, it should be appreciated that the system can be used for offering any type of medical treatment and consultation services.

The mobile cryotherapy service system 100 is used for scheduling cryotherapy sessions from a cryotherapy service provider using a computer-implemented software application 104 installed in an electronic device 102 such as a personal digital assistant (PDA), smartphone, laptop or any other such type of electronic handheld device. Similarly, the cryotherapy sessions can be scheduled using a website 106 accessed on a browser. The software application 104 and the website 106 are used for submitting scheduling requests to a cryotherapy service provider server 110. The electronic device 102 and the website 106 communicate with the server 110 over a communication network 108 such as the internet or through a private network.

In operation, when a user requests for a cryotherapy service treatment in the software application 104 or on the website 106, a booking request is sent to the cryotherapy service provider server 110 via a network such as the internet 108. On receiving the cryotherapy service treatment request, the cryotherapy service provider server 110 schedules a treatment session and provides the user an operator with a portable cryotherapy machine for his or her desired/remote location.

The portable machines for providing cryotherapy cause blood vessels and tissues to tighten and constrict. The portable machine delivers very cold carbon dioxide to the affected body area to produce effective therapeutic results and then subsequently gradually restores the body's temperature to a normal range and allows blood to return to normal circulatory pressure levels.

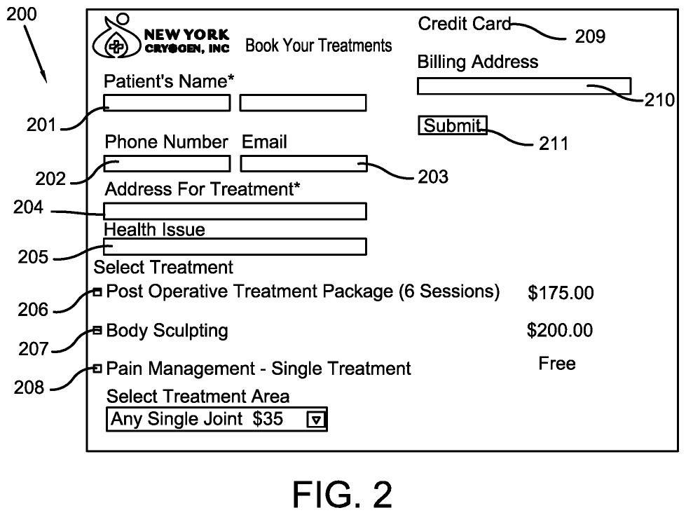

FIG. 2 illustrates a perspective view of an exemplary interface for booking cryotherapy treatments using cryotherapy service system of the present invention in accordance with the disclosed architecture. The user interface 200 allows a patient to input necessary details/information for booking a cryotherapy schedule and the information is submitted to the service provider server for processing. A user initially enters a patient's name 201. The user can enter the first name and the last name of the patient. The user enters a contact number 202, and an email address 203. The contact number 202 and the email address 203 are used for contacting and providing booking information to the patient. An address for treatment 204 is entered where the patient wants to schedule the cryotherapy session, and an operator with a portable cryotherapy machine is reserved for performing the treatment of the patient at the entered location. The patient's health problem can also be entered in the health issue parameter 205. The health issue 205 can be in the form of a drop-down menu having a list of relevant health issues from which a specific health issue can be selected for the patient.

A plurality of treatment options is displayed for a user to select from, where a user can select one or more treatments. As shown in FIG. 2, a plurality of treatments along with associated costs such as post-operative treatment package 206, body sculpting 207 and pain management 208 are shown as potential treatments. A user can select one or more treatments for which a session is scheduled at the address 204. Additionally, the user can select the date and time at which the cryotherapy session is to be scheduled.

The user interface 200 also enables the user to add payment details such as credit card information 209 for paying of the scheduled service. It should be appreciated that additional payment information such as a debit card or internet banking details can be added. A billing address 210 to which a physical invoice is shared can also be added on the user interface. Once all the information is entered, the booking request is submitted. The submitted request is stored on the service provider server and an operator for the scheduled session is assigned to perform the service.

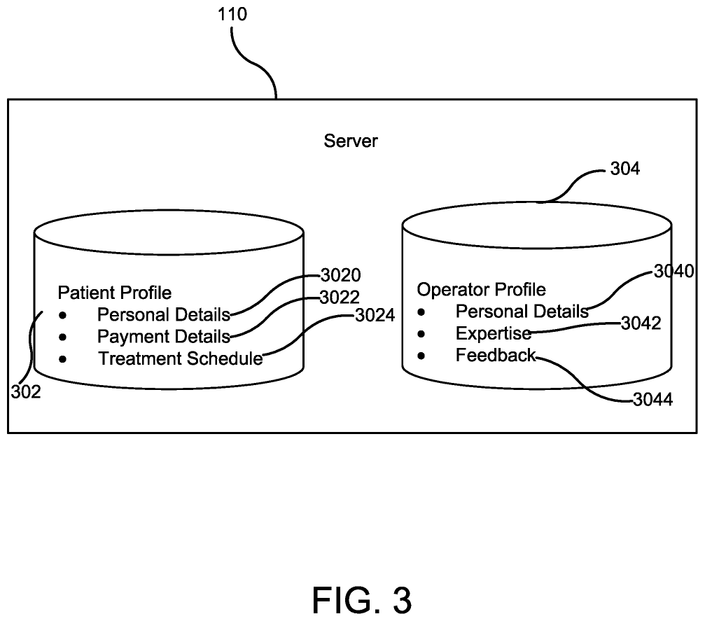

FIG. 3 illustrates storage of patient and operator information in a cryotherapy service provider server of the cryotherapy service system of the present invention in accordance with the disclosed architecture. The server 110 includes a patient profile database 302 that stores personal details 3020, payment details 3022, and treatment schedule details 3024 of the patients. The patient profile database 302 can be in the form of a relational database and may be coupled or integrated to the server 110.

Similarly, the server 110 stores information of the operators that perform a cryotherapy session with patients. An operator profile database 304 stores information such as personal details 3040 including name and contact details, expertise in treatments 3042 and feedback 3044 associated with the operators. The profile of an operator is used for assigning an operator for a treatment based on automatic matching of the treatment expertise 3042 with the treatment schedule 3024.

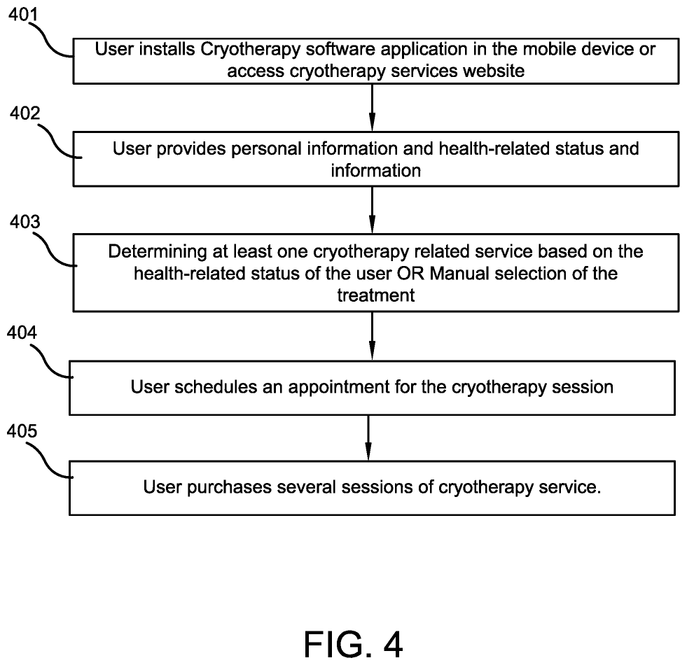

FIG. 4 illustrates a flow diagram showing exemplary steps performed by a patient for requesting cryotherapy services using the cryotherapy service system of the present invention in accordance with the disclosed architecture. Initially, a patient installs the mobile cryotherapy software application in one or more electronic devices or accesses the cryotherapy services website (Block 401). The patient can then provide personal information such as name, address for treatment, and health status-related information such as muscle aches and pains, inflammation, chronic pain or any other such type of health problems (Block 402). Thereafter, the cryotherapy service provider server can determine at least one cryotherapy-related service based on the health-related problem indicated by the patient (Block 403). Alternatively, the patient can select one or more treatments from a list of treatments shown on the user interface. The cryotherapy-related services can include post-operative treatments, body sculpting treatments, pain management treatments, dermatologic management treatments, Frotox treatments, and/or any other such type of cryotherapy service. The user, after selection of a treatment, schedules the cryotherapy sessions for reserving a portable treatment machine or device at the desired scheduled location (Block 404). Alternatively, an operator comes with, or delivers, the portable machine for the treatment of the patient. Once the cryotherapy session is scheduled, the user purchases the session of the cryotherapy service. Additionally, the user can also schedule and purchase a plurality of recurring cryotherapy sessions to consistently burn calories and boost metabolism (Block 405).

In one potential embodiment of the present invention, a 3M Tegaderm device can be used for the cryotherapy treatment. This medical-grade sheathing protects the skin and enables a user to aggressively treat the target body area while dramatically reducing the potential of burning or irritation.

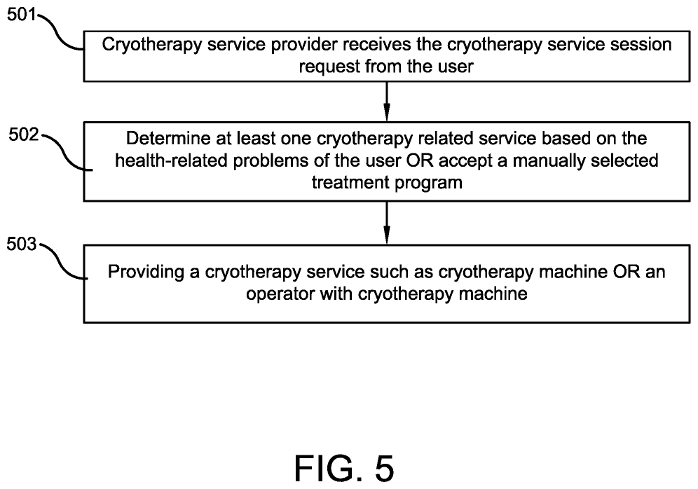

FIG. 5 illustrates a flow diagram showing steps performed by the cryotherapy service provider for providing cryotherapy service sessions to a user in accordance with the disclosed architecture. Initially, a cryotherapy service provider server receives a cryotherapy service session request from the user via a network such as the internet (Block 501). Then, the cryotherapy service provider server automatically determines at least one cryotherapy-related service based on the health-related problems of the user, or receives a predetermined selected treatment from the patient (Block 502). Then, the cryotherapy service provider server provides the user with a cryotherapy service session such as a cryotherapy machine for procurement and/or an operator with the desired cryotherapy machine for treatment at the scheduled address, date and time (Block 503).

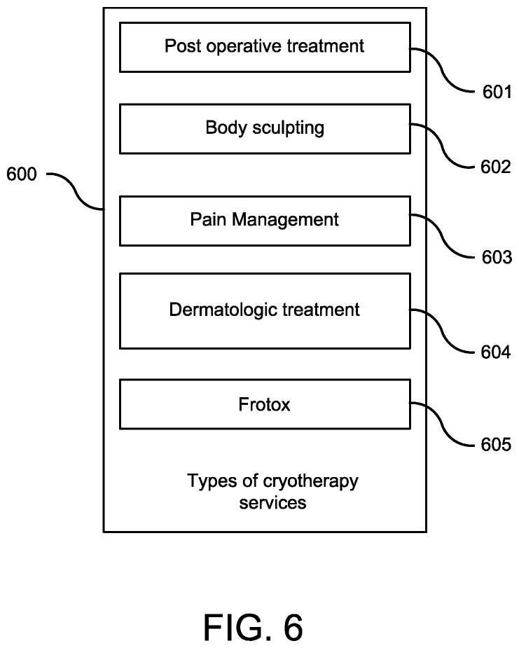

FIG. 6 illustrates a block diagram showing exemplary types of cryotherapy service sessions provided by cryotherapy service system of the present invention in accordance with the disclosed architecture. As shown in FIG. 6, the user is provided with the plurality of treatment options 600. The treatment options 600 include a post-operative treatment 601 which a patient can select after a surgical procedure. The post-operative treatment 601 meets the patient physical and psychological needs directly after surgery. A body sculpting treatment 602 facilitates shrinking of fat cells in the targeted areas of the body. Pain-management treatment 603 eases pain and suffering and improves the quality of life of those living with chronic pain. The pain management treatment option 603 provides users with a plurality of choices such as single joint, entire arm, upper back, hand and wrist, lumbar spine and other such types of choices related to pain management treatment.

The treatment option 600 can provide users with a dermatologic treatment 604 for the treatment of acne, eczema, and other such types of dermatologic treatments. The treatment option 600 can also include a Frotox treatment 605 for the treatment of the face, neck and scalp. The Frotox treatment 605 involves using a controlled stream of pressurized liquid nitrogen vapor applied to the face, neck and scalp. The Frotox treatment 605 produces an increased production of collagen and enhances skin firmness, while decreasing fine lines and wrinkles in the skin.

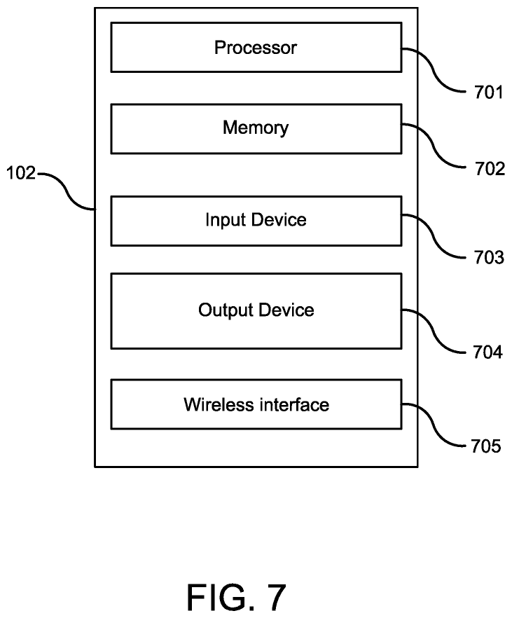

FIG. 7 illustrates an exemplary electronic device upon which the computer-implemented application of the present disclosure is implemented in accordance with the disclosed architecture. The electronic device 102 includes several components such as a processor 708 configured to perform one or more functions described herein in accordance with the computer-implemented instructions. The electronic device 102 includes input device(s) 703 such as a mouse, keyboard, touch input device, voice input device, etc. for entering data and information. The electronic device 102 also includes one or more output device(s) 704 such as a monitor, presence-sensitive display, or other display device.

The electronic device 102 includes a memory 702 used to store programs (sequences of instructions) or data (e.g. program state information) on a temporary or permanent basis for use. Memory 702 may be configured for short-term storage of information as volatile memory, and therefore not retain stored contents if powered off. Examples of volatile memories include random access memories (RAM), dynamic random access memories (DRAM), static random access memories (SRAM) and other forms of volatile memories known in the art.

The processor 701, in combination with one or more of memory 702, input device(s) 703 or output device(s) 704, can be utilized to provide potential patients with the ability to request or select cryotherapy sessions on a computer via the Internet. The connection to a network is provided by a wireless interface 705 allowing the patient to book cryotherapy sessions.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “cryotherapy service system”, “mobile cryotherapy service system” and “system” are interchangeable and refer to the cryotherapy service system 100 of the present invention.

Notwithstanding the forgoing, the cryotherapy service system 100 and any of its components can be of any suitable size and configuration as is known in the art without affecting the overall concept of the invention, provided that it accomplishes the above-stated objectives. One of ordinary skill in the art will appreciate that the size, configuration, and material of the cryotherapy service system 100 as shown in the FIGS. are for illustrative purposes only.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A computer implemented method for scheduling a cryotherapy service, the computer implemented method comprising the steps of:
accessing a cryotherapy services website;
entering a user's information including at least a patient's name, a contact number, an email address, a remote address, and a health status;
selecting a cryotherapy treatment;
scheduling an associated cryotherapy device for said cryotherapy treatment at said remote address, wherein said associated cryotherapy device is scheduled based on said treatment; and
scheduling an operator wherein said operator is trained on said associated cryotherapy device and said cryotherapy treatment.

2. The computer implemented method of claim 1, wherein said selecting said cryotherapy treatment is based on said health status.

3. The computer implemented method of claim 2 further comprising a step of purchasing said cryotherapy treatment.

4. The computer implemented method of claim 3 further comprising a step of delivering said associated cryotherapy device.

5. The computer implemented method of claim 4 further comprising a step of scheduling a plurality of recurring said cryotherapy treatments.

6. The computer implemented method of claim 5 further comprising a step of purchasing said plurality of recurring said cryotherapy treatments.

7. The computer implemented method of claim 6, wherein said cryotherapy treatment is selected from the group consisting of a post-operative treatment, a body sculpting treatment, a pain management treatment, a dermatologic treatment, and a Frotox treatment.

8. The computer implemented method of claim 6 further comprising a step of storing said user's information at said cryotherapy services website.

9. An electronic implemented method for scheduling a cryotherapy service, the electronic implemented method comprising the steps of:
accessing a cryotherapy services website using a computer implemented software application, wherein said computer implemented software application is installed on an electronic device and further wherein said electronic device is selected from a group consisting of a PDA, a smartphone and a laptop computer;
entering a patient's information including at least a name, a contact number, an email address, a remote address, and a health status;
selecting a cryotherapy treatment;
scheduling an associated cryotherapy device for said cryotherapy treatment at said remote address, wherein said associated cryotherapy device is scheduled based on said treatment; and
scheduling an operator wherein said operator is trained on said associated cryotherapy device and said cryotherapy treatment, wherein said cryotherapy treatment is selected from a group consisting of a post-operative treatment, a body sculpting treatment, a pain management treatment, a dermatologic treatment and a Frotox treatment.

10. The electronic implemented method of claim 9, wherein said selecting said cryotherapy treatment is based on said health status.

11. The electronic implemented method of claim 10 further comprising a step of purchasing said cryotherapy treatment.

12. The electronic implemented method of claim 11 further comprising a step of delivering said associated cryotherapy device.

13. The electronic implemented method of claim 12 further comprising a step of scheduling a plurality of recurring said cryotherapy treatments.

14. The electronic implemented method of claim 13 further comprising a step of purchasing said plurality of recurring said cryotherapy treatments.

15. The electronic implemented method of claim 14 further comprising a step of storing said patient's information at said cryotherapy services website.

16. An electronic implemented method for scheduling a cryotherapy service, the electronic implemented method comprising the steps of:
accessing a cryotherapy services website using a computer implemented software application, wherein said computer implemented software application installed in an electronic device and further wherein said electronic device is selected from a group consisting of a PDA, a smartphone and a laptop computer;
entering a patient's information including at least a name, a contact number, an email address, a remote address, and a health status;
selecting a cryotherapy treatment;
selecting said cryotherapy treatment is based on said health status;
scheduling an associated cryotherapy device for said cryotherapy treatment at said remote address, wherein said associated cryotherapy device is scheduled based on said treatment;
scheduling an operator, wherein said operator is trained on said associated cryotherapy device and said cryotherapy treatment and further wherein said cryotherapy treatment is selected from a group consisting of a post-operative treatment, a body sculpting treatment, a pain management treatment, a dermatologic treatment and a Frotox treatment;
storing said patient's information at said cryotherapy services website; and
purchasing said cryotherapy treatment.

17. The electronic implemented method of claim 16 further comprising a step of delivering said associated cryotherapy device.

18. The electronic implemented method of claim 17 further comprising a step of scheduling a plurality of recurring said cryotherapy treatments.

19. The electronic implemented method of claim 18 further comprising a step of purchasing said plurality of recurring said cryotherapy treatments.

20. The electronic implemented method of claim 16 further comprising a step of profiling said operators for assigning said operators with said associated cryotherapy device and said cryotherapy treatments.
